SEQUENCE LISTING (4 sequences)

SEQ ID NO: 1 - DNA, 57 bp, Artificial Sequence
  F1 primer
cccaagcttaatacgactcactatagggagacccaatcgccaccatggtgagcaagg

SEQ ID NO: 2 - DNA, 57 bp, Artificial Sequence
  R1 primer
gaagttgaaggatccggtaccgtcgacatgtcctcgatgttgtggcggatcttgaag

SEQ ID NO: 3 - DNA, 53 bp, Artificial Sequence
  F2 primer
atgtcgacggtaccggatccttcaacttccctgagggcggcgtgcagctcgcc

SEQ ID NO: 4 - DNA, 21 bp, Artificial Sequence
  R2 primer
tgatctagagtcgcggccgct

(19) United States
(12) Patent Application Publication
(10) Pub. No.: US 2015/0322403 A1
(43) Pub. Date: Nov. 12, 2015

(54) REGULATION OF EUKARYOTIC GENE EXPRESSION BY RIBOSOMAL READING-FRAME SWITCH EFFICIENCY CONTROL VIA REGULATORY ELEMENT UPSTREAM OF THE FRAMESHIFTING SITE

(71) Applicant: NATIONAL CHUNG HSING UNIVERSITY, Taichung (TW)

(72) Inventors: Kung-Yao Chang, Taichung (TW); Che-Pei Cho, Taichung (TW); Hsiu-Ting Hsu, Taichung (TW); Ya-Hui Lin, Taichung (TW)

(73) Assignee: NATIONAL CHUNG HSING UNIVERSITY, Taichung (TW)

(21) Appl. No.: 14/274,762

(22) Filed: May 11, 2014

(51) Int. Cl. C12N 5/071; C12N 5/04

(52) U.S. Cl. CPC C12N 5/0602; C12N 5/04

(57) ABSTRACT

A method of regulating gene expression in eukaryote includes administrating a ligand-sensing RNA element to regulate the formation of regulatory hairpin upstream of programmed ribosomal frameshifting (PRF) site. Further, a method of regulating ribosome frameshifting efficiency in the protein translation of a eukaryotic cell includes contacting the eukaryotic cell with a molecule to control the upstream programmed ribosomal frameshifting (PRF) regulatory duplex element formation, which does not involve messenger RNA degradation by RNase H or RNAi.

### BACKGROUND OF THE INVENTION

1. Technical Field of the Invention

The present invention relates to a method of regulating eukaryotic gene expression by targeting the regulatory element upstream of ribosomal frameshifting site. Especially, the present invention relates to a method of regulating gene expression in eukaryote by controlling duplex element formation immediately upstream of frameshifting site in programmed ribosomal frameshifting (PRF).

2. Description of the Related Art

The dynamic transition of alternative RNA conformations has been exploited by nature to regulate RNA-dependent cellular functions. This is achieved through regulatory formation of specific RNA structures embedded in distinct RNA-mediated functional platforms (Dethoff, E. A. et al., Nature 482, 322-330, 2012). Riboswitches, RNA elements responsive to metabolites, have been widely used to regulate transcription termination or translation initiation for tuning specific gene expressions in response to nutrient variations in prokaryotes (Henkin, T. M. Genes & Dev. 22, 3383-3390, 2008; Mandal, M. & Breaker, R. R. Nature Rev. Mol. Cell Biol. 5, 451-463, 2004).

Extensive efforts have been devoted to looking for chemical scaffolds capable of triggering riboswitch-mediated gene expression in both prokaryotic and eukaryotic systems (Deigan, K. E. et al., Acc. Chem. Res. 44, 1329-1338, 2011). Limited success in mammalian riboswitch applications may relate to mammalian systems using different mechanisms from those of prokaryotes to terminate transcription and initiate translation. The discovery of alternative splicing regulation by TPP riboswitches in fungi and plants (Cheah, M. T. et al., Nature 447, 497-500, 2007) suggests that other RNA-mediated gene-expression platforms may provide a better framework for constructing successful eukaryotic riboswitches.

The −1 programmed ribosomal frameshifting (PRF) causes an elongating ribosome to shift a single nucleotide in the 5′-direction of mRNA, leading to a −1 reading-frame switch during decoding. Efficient −1 PRF requires a slippery sequence (where frameshifting occurs) and an optimally placed downstream stimulator structure (usually a pseudoknot). By contrast, the +1 PRF causes an elongating ribosome to shift a single nucleotide in the 3′-direction of mRNA, leading to a +1 reading-frame switch during decoding (Farabaugh, P. J., Microbiol. Rev. 60, 103-134, 1996).

Recently, engineered metabolite-responsive RNA pseudoknots of prokaryotic origin were shown to possess ligand-specific −1 programmed ribosomal frameshifting (−1 PRF) stimulation activity in reticulocyte lysate. While the success of converting a ligand-responsive pseudoknot into a ligand-dependent −1 PRF stimulator suggests that translational reading-frame switch regulation holds promise as an expression platform for engineering mammalian riboswitches, its general application is hampered by the difficulty in finding specific ligand-responsive −1 PRF pseudoknots (Chou, M. Y. et al, RNA 16, 1236-1244, 2010; Yu, C. H. et al, ACS Chem. Biol. 8, 733-740, 2013).

The −1 PRF is crucial for a variety of human viral pathogens to replicate efficiently in the host and has been proposed as an antiviral target (Hung, M. et al., J. Virol. 72, 4819-4824, 1998). Viral −1 PRF stimulator structures downstream of frameshifting site were used as potential drug target by aiming to inhibit viral −1 PRF via ligands that block the function of −1 PRF stimulator. The ligands can be organic molecules that bind specifically to the stimulator structure (Park. S.-J. et al, J. Am. Chem. Soc. 133, 10094-10100, 2011) or antisenses sequence that disrupt the stimulator structures (Ahn, D.-G. et al, Antiviral Res. 91, 1-10, 2011). A potential drawback of using antisense to target downstream stimulator structure is that duplex formed between mRNA and antisense could stimulate −1 PRF by itself when placed downstream of the frameshifting site (Olsthoorn, R. C. L. et al, RNA 10, 1702-1703, 2004; Howard, M. T. et al. RNA 10, 1653-1661, 2004). We have previously shown that a hairpin upstream of the −1 PRF slippery site can attenuate −1 PRF efficiency. In this case, attenuation efficiency is determined by hairpin stability and its distance from the slippery site. Additionally, the hairpin was also capable of stimulating +1 PRF in yeast when placed upstream of a +1 frameshifting site, suggesting a proximal stable hairpin upstream of frameshifting site is indeed a regulator of ribosomal reading-frame switch. (Cho, C. P. et al., PLoS ONE 8, e62283, 2013).

It is noted that this co-translational refolding RNA hairpin was reminiscent of co-transcriptional folding RNA hairpins that modulate the ρ-independent transcriptional termination efficiency in prokaryotic systems, and reasoned that the regulatory hairpin upstream of frameshifting site might be regulated in ways similar to hairpins in the prokaryotic transcription termination. This means ligand-dependent regulation of the upstream attenuator hairpin formation provides an alternative way in building ligand-responsive −1 PRF regulatory circuits in addition to using a downstream ligand-responsive pseudoknot stimulator.

Attempt to dissect the role of upstream hairpin in ribosomal reading-frame switch regulation revealed that an antisense complementary to the mRNA sequence upstream of the −1 PRF frameshifting site also down-regulated −1 PRF activity, suggesting the reformed hairpin stem is the functional determinant of frameshifting regulation. Thus, a proximal stable hairpin in mRNA as well as a proximal duplex mediated by a trans-acting antisense can function as reading-frame switch regulator when placed upstream of the frameshifting site. In contrast, short duplex mediated by the internal Shine-Dalgarno (SD)-antiSD interaction between mRNA and prokaryotic 16S ribosomal RNA upstream of frameshifting site can stimulate −1 and +1 PRF in prokaryotic cell (Weiss, R. B. et al, EMBO J. 7, 1503-1507, 1988; Larsen, B. et al, J. Bacteriol. 176, 6842-6851, 1994). However, there is no SD-antiSD interaction in eukaryotic translational systems.

### SUMMARY OF INVENTION

Accordingly, in one aspect, the present invention provides a method of regulating eukaryotic gene expression by targeting upstream regulatory element of the frameshifting site, which comprises controlling duplex formation upstream of the programmed ribosomal frameshifting site.

In some embodiments of the present invention, the method of regulating gene expression in a eukaryotic cell comprises administrating a ligand-sensing RNA element to regulate the formation of upstream −1 programmed ribosomal frameshifting attenuator hairpin. In some embodiments of the present invention, the eukaryotic cell is a plant cell. In other embodiments of the present invention, the eukaryotic cell is a mammalian cell.

In some embodiments of the present invention, the regulation of upstream PRF regulatory hairpin formation includes an enhancement or inhibition of the formation of the hairpin structure. In one embodiment of the present invention, the programmed ribosomal frameshifting is −1 PRF. In another embodiment of the present invention, the programmed ribosomal frameshifting is +1 PRF.

In one embodiment of the present invention, the reading-frame switch regulation can be achieved by an antisense sequence complementary to the mRNA sequence upstream of frameshifting site to form an upstream duplex.

In one embodiment of the present invention, the ligand is an anti-sense sequence complementary to the upstream PRF regulatory hairpin forming sequence in mRNA of the gene. In another embodiment of the present invention, the ligand is a molecule binding to the upstream PRF regulatory hairpin forming sequence in mRNA of the gene.

In a further embodiment of the present invention, the ligand is a RNA-binding protein. In a further embodiment of the present invention, the ligand is an organic compound binding to the upstream PRF regulatory hairpin forming sequence in mRNA of the gene.

In another aspect, the present invention relates to a method for regulating ribosome frameshifting efficiency in the protein translation of a eukaryotic cell, which comprises contacting the eukaryotic cell with a molecule to regulate formation of the upstream regulatory duplex element. In some embodiments of the present invention, the eukaryotic cell is a plant cell. In other embodiments of the present invention, the eukaryotic cell is a mammalian cell.

In one embodiment of the present invention, the molecule is a RNA-binding protein. In another embodiment of the present invention, the molecule is an organic compound.

### BRIEF DESCRIPTION OF THE DRAWINGS

FIG. 1A presents a scheme showing that a stable hairpin upstream of the slippery site and a downstream pseudoknot stimulator possess opposite effects on −1 programmed ribosomal frameshifting activity, while the upstream hairpin formation might be regulated by a nearby ligand-sensing RNA element. FIG. 1B presents a scheme showing some possible ligands which may be used as the regulator to control duplex formation (reformed stem of a hairpin or an antisense-mediated duplex) upstream of programmed ribosomal frameshifting site.

FIG. 2 presents a model showing that both RNA conformation dynamics and RNA-ligand complex formation are modulated by the elongating ribosome with at least four different stages.

FIG. 3A shows the sequence designs and illustrations for RNA-protein interactions mediated −1 PRF attenuator hairpin disruption and promotion in GlcT-OFF and GlcT-ON RNA elements, respectively. Mfold was used to predict structures and free energies of the attenuator and anti-attenuator hairpins. FIG. 3B shows the in vitro radioactivity-based −1 PRF activity analysis of a potent downstream DU177 pseudoknot stimulator with an upstream GlcT-OFF or GlcT-ON element using a shortened p2luc reporter in the presence of increasing amounts of GlcTRBD in reticulocyte lysate. FIG. 3C shows the relative −1 PRF activity of reporter constructs in FIG. 3B in the presence of different dosages of GlcTRBD with GlcTRBD-free activity being treated as 1 (in grey).

FIG. 4A shows the sequences, predicted structures and free energies of theo-OFF1, theo-mOFF1 and theo-OFF2. The boxed nucleotides were deleted in the negative control theo-mOFF1 element. FIG. 4B shows in vitro radioactivity-based −1 PRF activity analysis of a potent downstream DU177 pseudoknot stimulator with an upstream theo-OFF1 or theo-OFF2 element using a shortened p2luc reporter in the presence of increasing amounts of theophylline in reticulocyte lysate. FIG. 4C shows the relative fold change in −1 PRF activity for reporter constructs in FIG. 4B in the presence of different dosages of theophylline compared with the activity of constructs without theophylline addition (in grey).

FIG. 5A shows the sequences, predicted structures and free energies of theo-ON. FIG. 5B shows in vitro radioactivity-based −1 PRF activity analysis of a potent downstream DU177 pseudoknot stimulator with an upstream theo-ON element using a shortened p2luc reporter in the presence of increasing amounts of theophylline in reticulocyte lysate. FIG. 5C shows the relative fold change in −1 PRF activity for reporter constructs in FIG. 5B in the presence of different dosages of theophylline compared with the activity of constructs without theophylline addition (in grey).

FIG. 6A shows the radioactivity-based −1 PRF activity analysis of a potent downstream DU177 pseudoknot stimulator with an upstream theo-OFF2 or control element using p2luc reporter in the presence of increasing amounts of theophylline with the wheat germ extract being used as the in vitro translation system. FIG. 6B shows relative fold change in −1 PRF activity for reporter constructs in FIG. 6a in the presence of different dosages of theophylline compared with the activity of constructs without theophylline addition (in grey).

FIG. 7A shows the upstream theo-OFF element working with an SAH-dependent −1 PRF stimulator to act as a two-input logic gate in 293T cells. FIG. 7B shows the relative −1 PRF activity of 293T cells transfected with a full-length p2luc −1 PRF reporter harboring the SAH-responsive pseudoknot stimulator and the upstream theo-OFF2 element in the presence of different amounts of theophylline and Adox (adenosine-2′,3′ dialdehyde, a cell-permeable AdoHcy hydrolase inhibitor that blocks SAH hydrolysis to enhance intracellular SAH concentration). The activity of constructs without theophylline and Adox addition was treated as 1 (in grey). The −1 PRF activity was calculated from dual-luciferase activity measured in 293T cells, transfected with different −1 PRF constructs.

FIG. 8A shows the scheme for using the N- and C-terminal parts of splited Venus to track the expression of 0- and −1 frames, respectively. FIG. 8B shows fluorescence microscopy images of 293T cells, transfected with a pN insert C-Venus −1 PRF reporter harboring an upstream theo-OFF2 element and downstream SAH-responsive pseudoknot in the linker region, in the presence of different amounts of theophylline and Adox. (Scale bar, 10 m). FIG. 8C shows Western blot results of 293T cell lysates from cells transfected with the −1 PRF report in FIG. 8b.

FIG. 9A shows the radioactivity-based −1 PRF activity analysis of an SAH-sensing pseudoknot stimulator with an upstream theo-OFF2 (right panel) or control element (left panel) using p2luc reporter in the presence of different amounts of theophylline and SAH with the wheat germ extract being used as the in vitro translation system. FIG. 9B shows the relative fold change in −1 PRF activity for reporter construct lacking the theo-OFF2 element in the presence of different amounts of theophylline and SAH, compared with the activity of constructs without ligand addition (in grey). FIG. 9C shows the relative fold change in −1 PRF activity for reporter construct carrying the theo-OFF2 element in the presence of different amounts of theophylline and SAH, compared with the activity of constructs without ligand addition (in grey).

FIG. 10A shows a schematic drawing of the immediate hairpin-forming sequences upstream of −1 PRF slippery site in constructs used and the designed antisense DNA blockers. The targeted sequences in the construct are underlined and sequences corresponding to the 5′- and 3′-half of the −1 PRF attenuator hairpin stem (6BPGC) are typed boldly, whereas the sequences of the antisense blocker are typed in lowercase. FIG. 10B shows the SDS-PAGE analysis of the −1 PRF assay of 6BPGC containing reporter in the presence of corresponding antisense DNA blocker. The concentrations of DNA blockers and the translated proteins corresponding to the 0 frame and −1 frame products are labeled as indicated. FIG. 10C shows the relative frameshifting activity of 6BPGC containing reporter in the presence of corresponding antisense blocker, with the frameshifting efficiency of the reporter alone treated as 100% (in grey).

FIG. 11A shows a schematic drawing of the immediate sequences upstream of −1 PRF slippery site in constructs used and the designed antisense DNA oligonucleotide. The targeted upstream sequences (6BPGC5′WT) in the construct are underlined, whereas the sequences of the antisense oligonucleotide are typed in lowercase. FIG. 11B shows the SDS-PAGE analysis of the −1 PRF assay of 6BPGC5′-WT containing reporter in the presence of corresponding antisense DNA oligonucleotide. The concentrations of DNA oligonucleotide and the translated proteins corresponding to the 0 frame and −1 frame products are labeled as indicated. FIG. 11C shows the relative frameshifting activity of 6BPGC5′-WT containing reporter in the presence of corresponding antisense oligonucleotide, with the frameshifting efficiency of the reporter alone treated as 100% (in grey).

FIG. 12A shows the distance effects of an upstream duplex mediated by antisense DNA on −1 PRF attenuation efficiency. The sequence of the antisense DNA used and the predicted free energy of the resulted DNA-RNA duplex are shown in FIG. 12a. The duplex mediated by T1-0 oligonucleotide possesses no spacing from the UUUAAAC slippery site, while the duplex mediated by oligonucleotide T1-1 possesses 1 nucleotide spacing from the slippery site and so on. FIG. 12B shows the relative −1 PRF frameshifting efficiency in the presence of an antisense-mediated upstream RNA-DNA duplex with different spacing toward the frameshifting site. The results indicate that a stable upstream duplex need to be close to the frameshifting site to be an effective −1 PRF attenuator.

FIG. 13A shows the sequence covering the −1 PRF signal in NS2A gene of West-Nile virus (WNV) (Melian, E. B. et al, J. Viorl. 84, 1641-1647, 2010) cloned into the p2luc −1 PRF reporter with the predicted stimulator pseudoknot structure. The upstream WNV viral sequence, targeted by the anti-WNV antisense DNA blocker is typed in bold and the slippery site is underlined, while the sequence of antisense DNA blocker is typed in lowercase. FIG. 13B shows the relative frameshifting activity of the p2luc-WNV −1 PRF reporter in the presence of different amounts of anti-WNV DNA and control oligonucleotide. The frameshifting efficiency of the reporter alone was treated as 100% (in grey) for comparison based on dual-luciferase assay of −1 PRF activity in reticulocyte lysate. Error bars, s.d.; n=3.

FIG. 14A shows a schematic drawing for the p2luc-based yeast −1 PRF reporter construct containing upstream theo-OFF2 element followed by a yeast +1 frameshifting site. Please note that an upstream hairpin acts as a +1 frameshifting stimulator and addition of theophylline to disrupt upstream hairpin formation will lead to the down-regulation of +1 frameshifting activity. FIG. 14B shows fold change of the CUUUGG dependent +1 frameshifting activity based on dual-luciferase assay in the presence of different amounts of theophylline and caffeine using the activity of theophylline-free condition as 1 in yRP1674 yeast cells (Mat a his3Δ1 leu2Δ met15Δ ura3Δ). Error bars, s.d.; n=6.

### DETAILED DESCRIPTION OF THE INVENTION

The characteristics and advantages of the present invention will be illustrated and described in detail in the following examples. The examples described herein are intended to be illustrations, not limitations of the invention.

Prokaryotic transcriptional termination can be triggered by a co-translational folding GC-rich terminator hairpin with a downstream U-stretch sequence in the transcribed RNA and be further regulated by a ligand-sensing element upstream of the terminator hairpin.

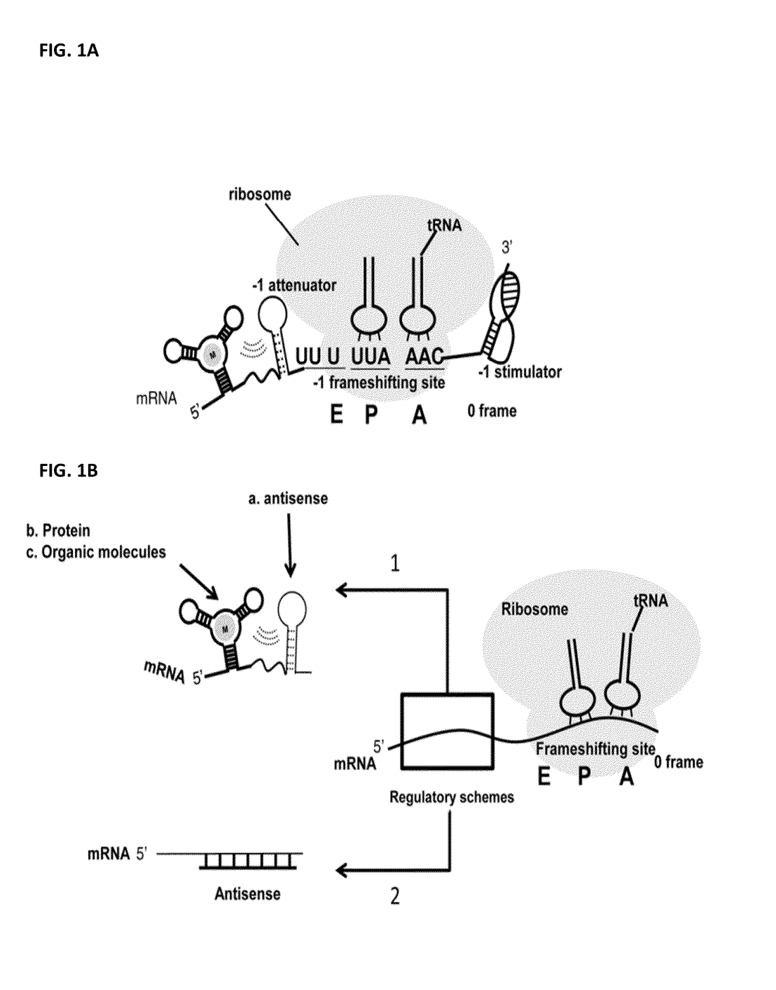

In Eukaryotic systems, it has been suggested that a co-translational refolding hairpin upstream of the slippery site and a downstream pseudoknot stimulator possess opposite effects on −1 programmed ribosomal frameshifting activity (FIG. 1A). This co-translational refolding RNA hairpin was reminiscent of co-transcriptional folding RNA hairpins that modulate the ρ-independent transcriptional termination efficiency in prokaryotic systems (Santangelo, T. J. and Artsimovitch, I. Nature Rev. Microbiol. 9, 319-329, 2011). It is reasoned that this −1 PRF attenuator hairpin might be regulated in ways similar to hairpins in the prokaryotic transcription termination.

FIG. 1B shows some kinds of possible ligand which may be used as the regulator of upstream programmed ribosomal frameshifting regulatory hairpin formation, which does not involve messenger RNA degradation by RNase H or RNAi.

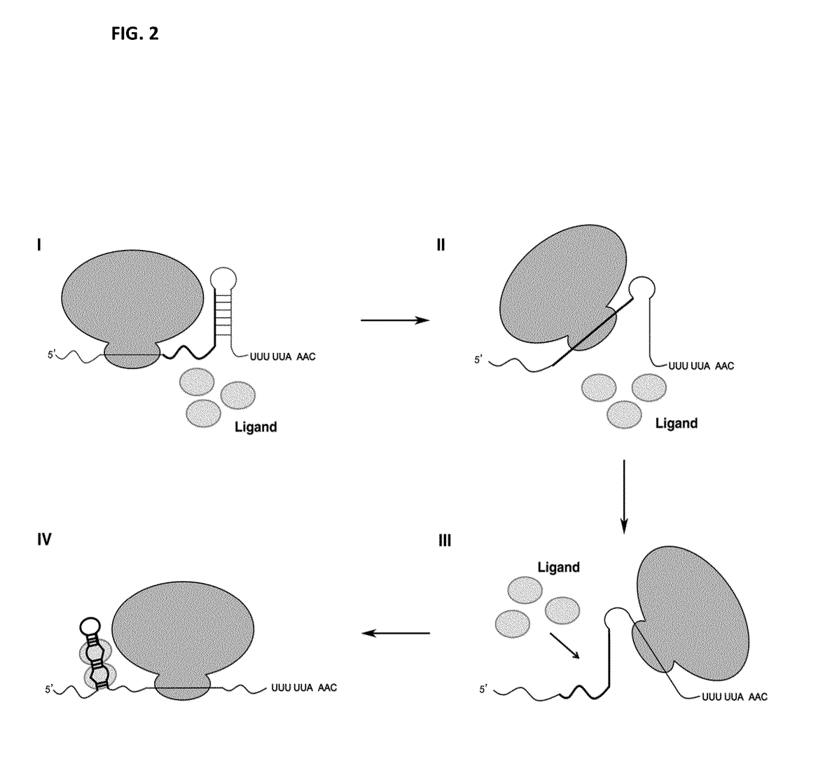

In this context, we reasoned that the elongating ribosome would help unwind the regulatory hairpin stem that traps the 3′-side of ligand-responsive element and facilitate transient RNA-ligand interaction as illustrated in the model in FIG. 2.

In FIG. 2, a model showing that both RNA conformation dynamics and RNA-ligand complex formation are modulated by the elongating ribosome with at least four different stages. It suggests that the unwinding of the attenuator hairpin stem by ribosome (stage II) will help release the trapped 3′-component of ligand-sensing RNA element (stage III) to facilitate RNA-ligand complex formation (stage IV), and thus interfere with regulatory hairpin refolding when the same ribosome occupies the frameshifting slippery site. Strictly speaking, the potential involvement of ribosome in RNA rearrangement processes makes this system not fit to the definition of a riboswitch (Henkin, T. M. Genes & Dev. 22, 3383-3390, 2008) and be more similar to trp operon attenuation (Yanofsky, C. J. Bacteriol. 182, 1-8, 2000).

−1 PRF elements containing different combination of pseudoknot stimulators and upstream attenuator sequences were chemically synthesized and purchased from Mission Biotech, Taiwan. Longer elements were constructed by assembling different pieces of chemically synthesized DNA oligo-nucleotides with partially overlapping sequences via the PCR-based ligation approach.

Control of Upstream −1 PRF Attenuator Hairpin Formation by RNA-Protein Interaction.

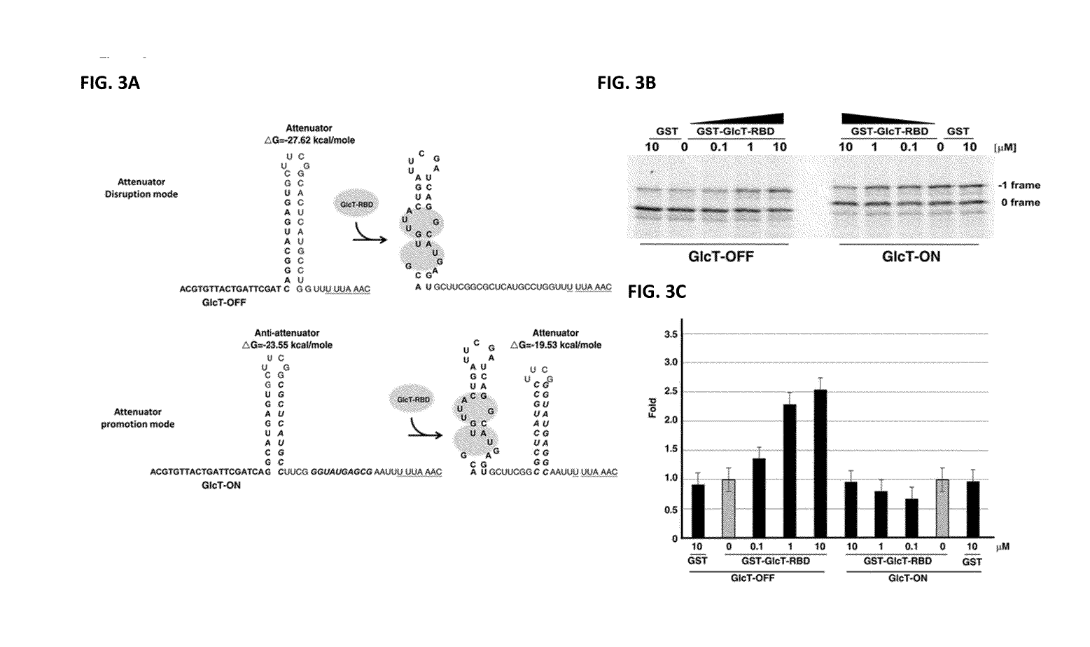

As the ribonucleic-antiterminator (RAT) sequence of Bacillus subtilis ptsGHI operon adopts an unstable internal loop conformation and can be stabilized by the binding of GlcT antiterminator protein (Langbein, I. et al, J. Mol. Biol. 293, 795-805, 1999), we designed a chimeric RNA element containing an RAT sequence (GlcT-OFF) with the 3′-side of RAT internal loop embedded in a stable hairpin stem and placed the chimeric RNA element upstream of the slippery site with a potent downstream −1 PRF stimulator (FIG. 3A). We rationalized that the engineered hairpin is the dominant conformation and serves as an upstream −1 PRF attenuator, whereas the addition of GlcT protein should disrupt the attenuator hairpin via RAT-GlcT complex formation and thus restore −1 PRF activity. In this context, a recent ribosome profiling study has indicated that mammalian ribosomes are well spaced during translation (Ingolia, N. et al, Cell 147, 789-802, 2011), suggesting that the designed protein-mediated RNA conformation rearrangement could be achieved co-translationally.

The in vitro-1 PRF efficiency of a reporter containing the designed upstream chimeric element (GlcT-OFF) was up-regulated upon the addition of purified GlcT protein in a dosage-dependent manner (see, FIGS. 3B and 3C). We then designed the second chimera to down-regulate the −1 PRF by the formation of an efficient attenuator hairpin promoted by the same RNA-protein interactions (FIG. 3A).

The 5′-side of attenuation hairpin and the 3′-side of RAT internal loop are adjusted to form a stable hairpin (the anti-attenuator) that does not act as a −1 PRF attenuator due to its distant spacing from the slippery site. Therefore, the GlcT-ON RNA element is embedded with three potential RNA motifs; that is, an RAT internal loop, an anti-attenuator hairpin and an efficient −1 PRF attenuator hairpin with the component of the anti-attenuator hairpin being overlapped with the other two motifs. Without the GlcT protein, this anti-attenuator hairpin might be the dominant conformation. Upon GlcT protein addition, the equilibrium might be driven by RNA-protein interaction to disrupt the anti-attenuator hairpin stem and thus release its 3′-side to facilitate the formation of an efficient −1 PRF attenuator hairpin.

Consistently, the in vitro-1 PRF activity of a reporter containing the second chimeric element (GlcT-ON) was repressed by purified GlcT protein in a dosage-dependent manner (FIGS. 3B and 3C). Together, these proof-of-principle experiments indicate that −1 PRF activity can be regulated in-trans via a set of RNA-protein interactions designed to control the formation of an upstream −1 PRF attenuation hairpin.

Control of Upstream −1 PRF Attenuator Hairpin Formation by Organic Molecules

In a further embodiment, theophylline is used as a regulatory module based on small ligand-RNA interactions for exploring the possibility of replacing the RAT-GlcT interaction with the theophylline aptamer-theophylline interaction.

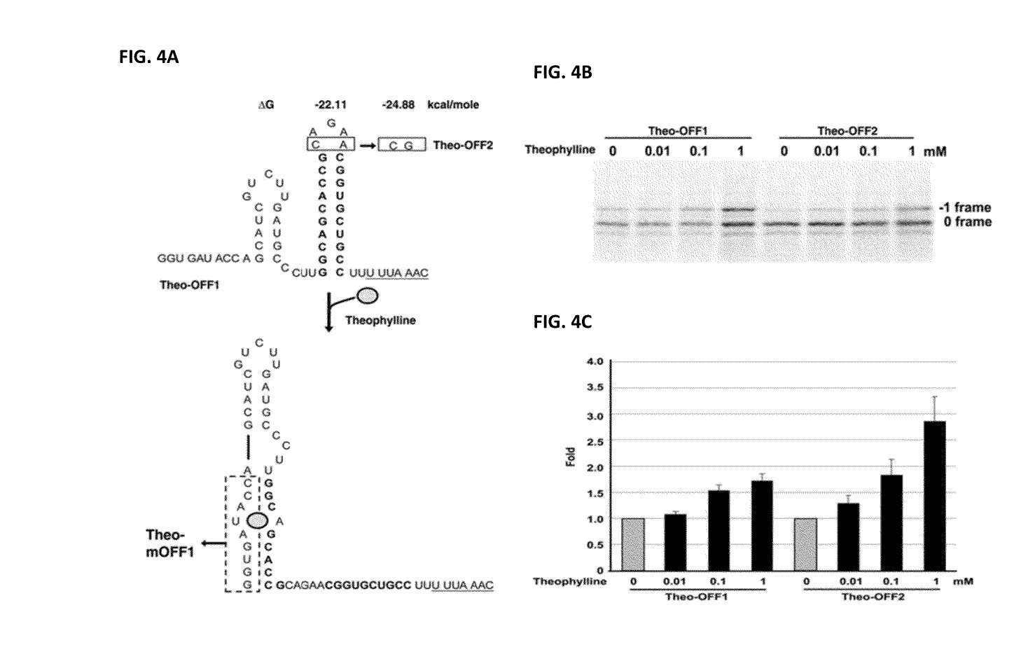

To this end, a designed −1 PRF attenuation hairpin was constructed with its 5′-stem sequences being complementary to the 3′-side sequences of a high affinity theophylline aptamer (FIG. 4A). Similar to RAT-GlcT dependent regulation, the in vitro −1 PRF activity of a DU177 pseudoknot stimulator driven reporter, harboring this designed upstream theo-OFF1 element, was up-regulated upon theophylline addition in a dosage-dependent manner (FIG. 4B and FIG. 4C).

We then modified the sequence in the attenuation hairpin loop of theo-OFF1 element without changing the theophylline-RNA interaction, and found that the dynamic range could be improved further by stabilizing the attenuator hairpin (see the theo-OFF2 element in FIG. 4).

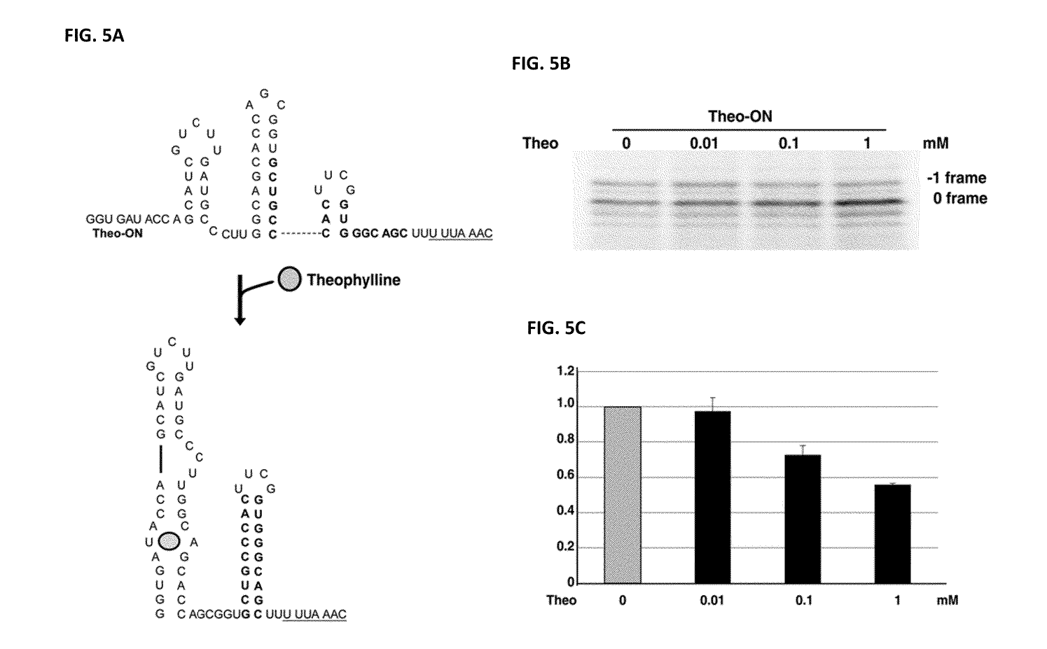

A theo-ON element was then designed to build a theophylline-dependent −1 PRF attenuator hairpin promotion mode that led to the down-regulation of −1 PRF activity in a theophylline-dependent manner (FIG. 5).

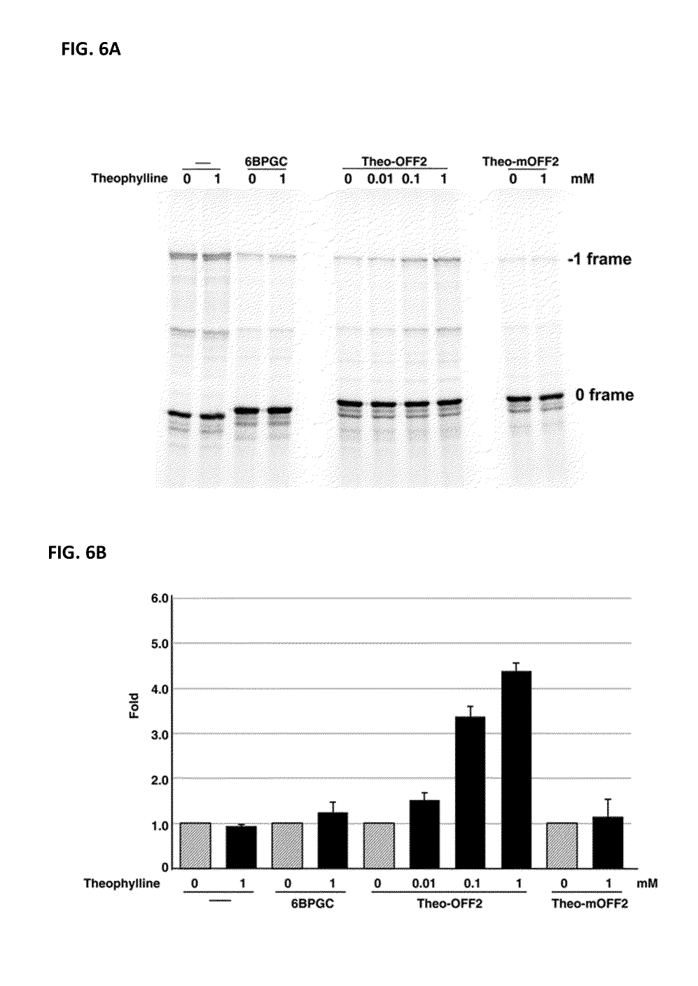

FIG. 6 shows the dynamic range of theophylline-dependent regulation of −1 PRF by theo-OFF2 is enhanced further in wheat germ lysates. Interestingly, an even higher dynamic range of theophylline-dependent −1 PRF activity was observed in wheat germ lysate, suggesting this −1 PRF regulatory module may also work in plants.

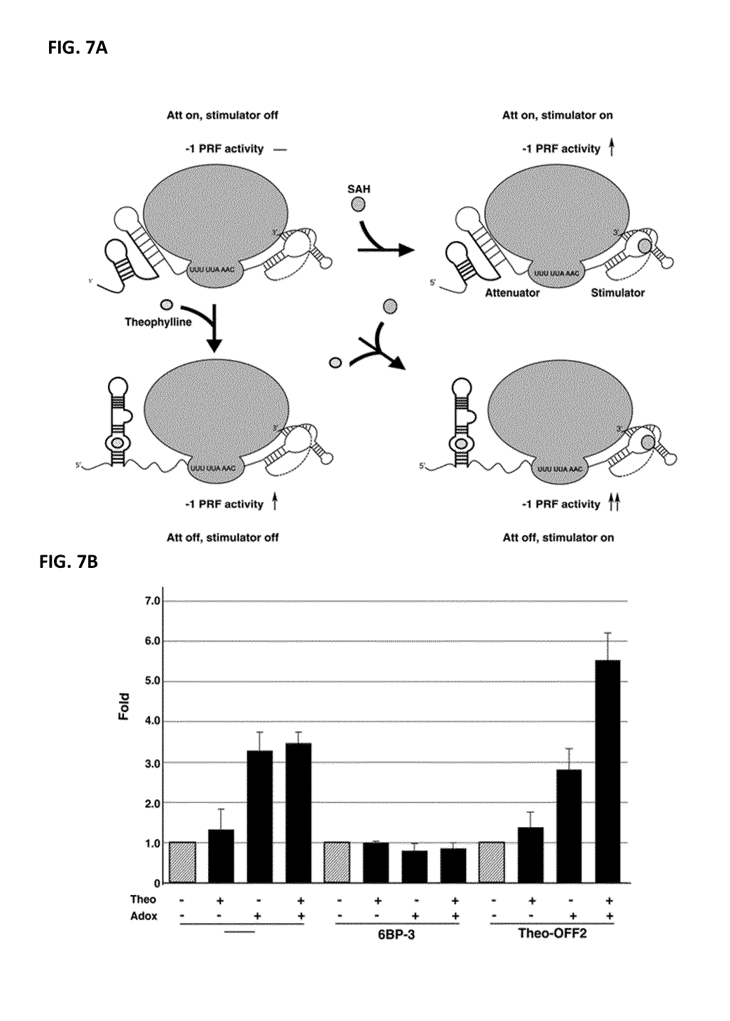

The ability to regulate −1 PRF in opposite directions by small molecule-induced conformational rearrangements of the attenuator and stimulator (FIG. 7A) provides a unique opportunity to build a two-input logic gate using the −1 PRF as platform.

We then fused theo-OFF2 with an SAH-dependent −1 PRF stimulator and examined the ligand-dependent −1 PRF activity in mammalian cells. The −1 PRF activity was close to the background level without the addition of theophylline and denosine-2′,3′-dialdehyde (Adox), a cell-permeable AdoHcy hydrolase inhibitor that blocks SAH hydrolysis to enhance intracellular SAH concentration, while the addition of theophylline or Adox only mildly increased −1 frameshifting efficiency in 293T cells. By contrast, the addition of both theophylline and Adox enhanced −1 frameshifting in a synergetic way (FIG. 7B).

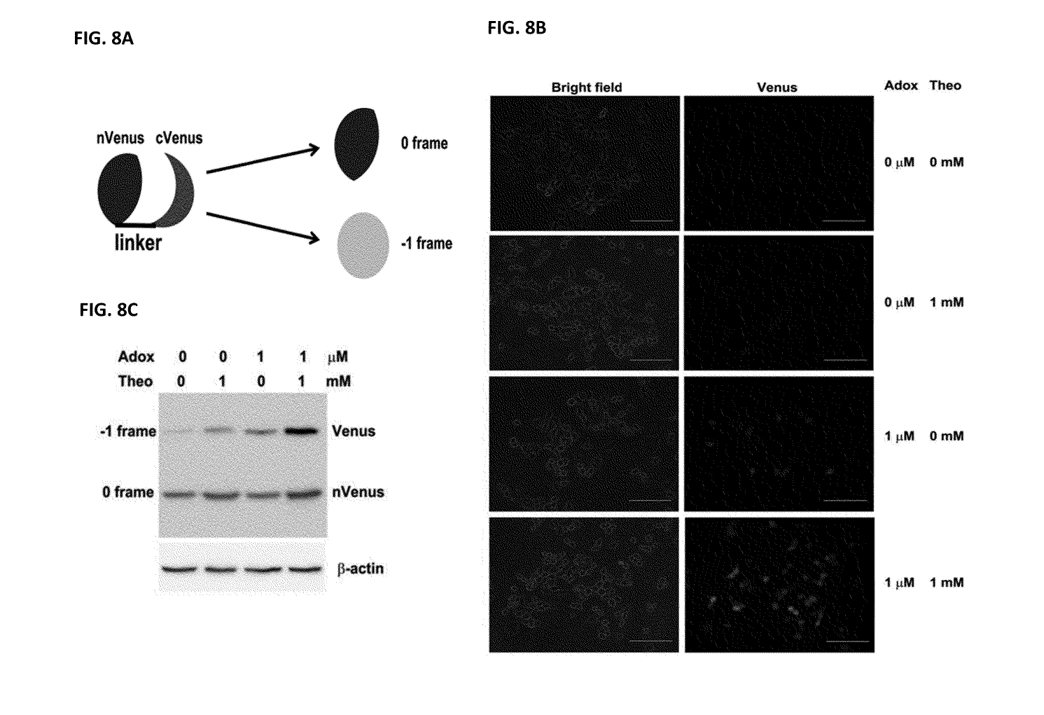

In a further embodiment, an alternative two-input regulatory −1 PRF switch was engineered based on a modified bimolecular fluorescence approach (Citovsky, V. et al, J. Mol. Biol. 362, 1120-1131, 2006). As separated N-terminal and C-terminal domains of YFP work in-trans to reconstitute a functional fluorescent protein, we used the −1 PRF switch to link the split N and C domains of Venus (derived from YFP) with the coding region of C Venus being shifted to the −1 frame. Thus, in this approach, Venus activity observation would be an indication of −1 frameshifting (FIG. 8A).

To generate a Venus-based reporter suitable for −1 PRF activity analysis in 293T cell, gene fragment encoding N-Venus (residues 1-174) or C-Venus (residues 175-240) was amplified using pNPY-Venus-N1 (provided by Professor A. Miyawaki at RIKEN) as the template. Primers F1 and R1 were used to amplify N-Venus to create an extended C-terminal linker with its terminal 29 nucleotides sequences (underlined) overlapped with nucleotide sequences of the extended N-terminal linker of the C-Venus fragment amplified by primers F2 and R2. The underlined complementary region in R1 and F2 primers both contain SalI and BamHI restriction sequences (typed boldly) that can provide the insertion site for frameshifting element cloning in the fluorescent −1 PRF reporter. The two amplified gene fragments were then fused by the PCR-based ligation approach using F1 and R2 as primers to generate a composite gene fragment with a cloning sites embedded linker sequence connecting nucleotide sequences corresponding to residues 174 and 175 of the original Venus ORF. The linker inserted Venus fragments were then used to replace the original Venus in pNPY-Venus-N1 to generate a vector pNinsertC-Venus.

F1:
(SEQ ID NO: 1)
5′-CCCAAGCTTAATACGACTCACTATAGGGAGACCCAATCGCCACCATG
GTGAGCAAGG-3′
R1:
(SEQ ID NO: 2)
5′-GAAGTTGAAGGATCCGGTACCGTCGACATGTCCTCGATGTTGTGGCG
GATCTTGAAG-3′
F2:
(SEQ ID NO: 3)
5′-ATGTCGACGGTACCGGATCCTTCAACTTCCCTGAGGGCGGCGTGCAG
CTCGCC-3′
R2:
(SEQ ID NO: 4)
5′-TGATCTAGAGTCGCGGCCGCT-3′

We found that prominent Venus activity could be observed in the presence of both theophylline and Adox, as showed in FIG. 8B and FIG. 8C. Thus, these experiments clearly demonstrate the potential of −1 PRF activity regulation in building a two-input logic gate by using small ligands that modulate the RNA conformations of two RNA motifs of opposite role in −1 PRF.

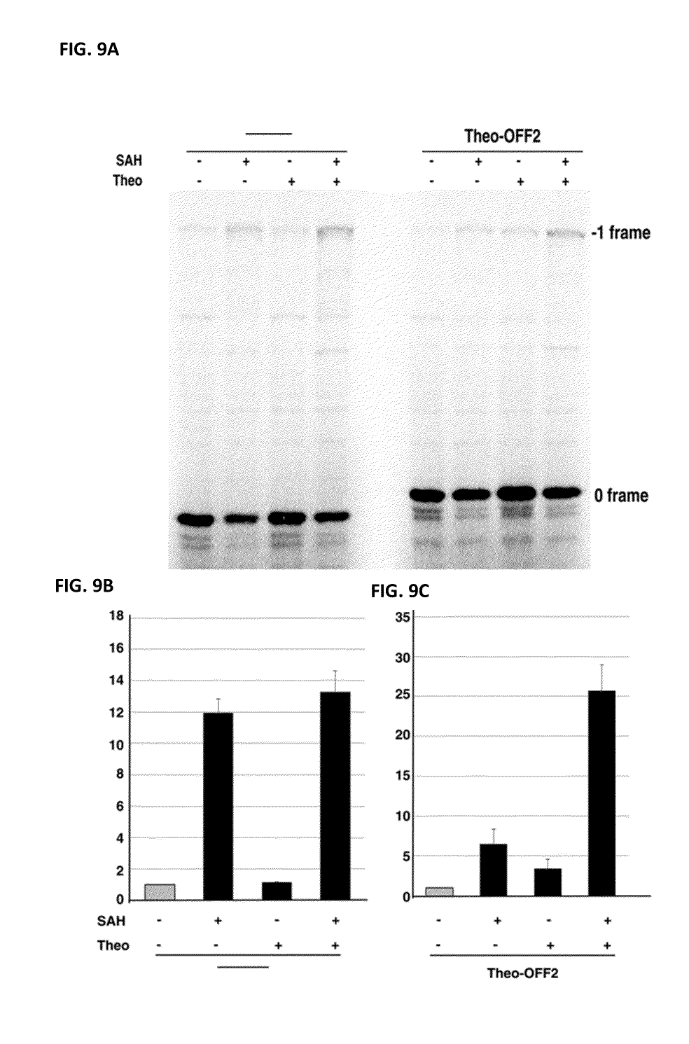

We also fused theo-OFF2 with an SAH-dependent −1 PRF stimulator and examined the ligand-dependent −1 PRF activity in vitro using wheat germ lysate. The −1 PRF activity was close to the background level without the addition of theophylline and SAH, while the addition of theophylline or SAH only mildly increased −1 frameshifting efficiency. By contrast, the addition of both theophylline and SAH enhanced −1 frameshifting in a synergetic way (FIG. 9).

Design of Anti-Sense DNA Oligonucleotide Targeting the Upstream Hairpin Stem for −1 PRF Frameshifting Activity Regulation

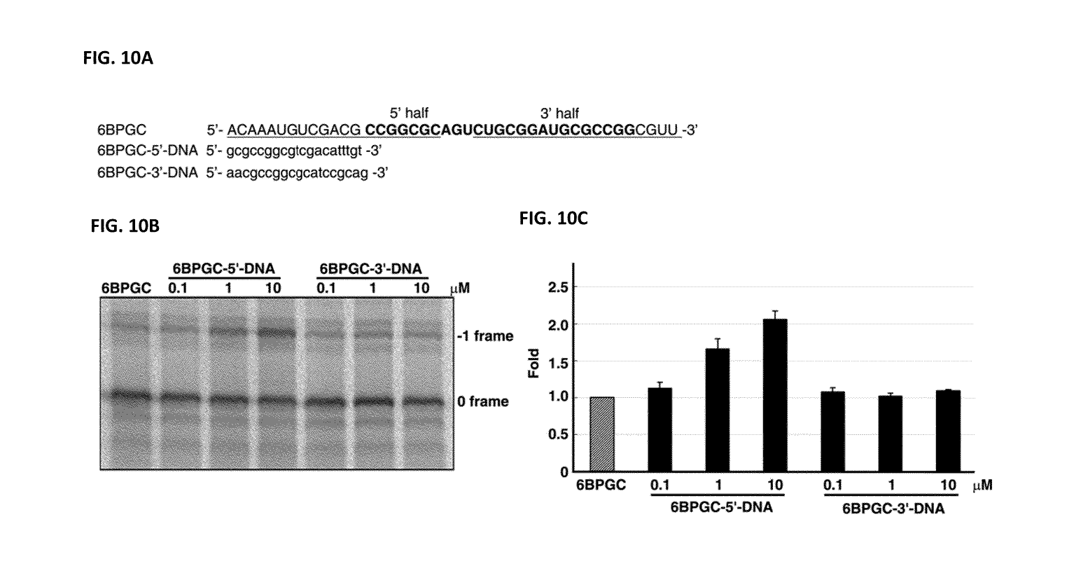

As one kind of ligands for regulation, DNA oligonucleotide with sequences complementary to the 5′-half (6BPGC-5′-DNA) or the 3′-hlaf (6BPGC-3′-DNA) sequences of a potent −1 PRF attenuator hairpin (6BPGC) stem (FIG. 10A) are designed, and further measured their effects on attenuation efficiency of a −1 PRF reporter containing the hairpin. Interestingly, addition of 6BPGC-5′-DNA resulted in a dose-dependent loss of the 6BPGC attenuator activity, whereas addition of 6BPGC-3′-DNA did not suppress attenuator activity at all (FIGS. 10A, 10B and 10C). Based on the critical effect of proximity on attenuation efficiency of a cis-acting attenuator hairpin (Cho, C. P. et al, PLoS ONE 8, e62283, 2013), this discrepancy could be caused by the different spacing from the slippery site between the two RNA-DNA duplexes formed.

Upstream Proximal Duplex as a Functional Unit for −1 PRF Attenuation and Distance Effect of Upstream Duplex on −1 PRF Attenuation Efficiency.

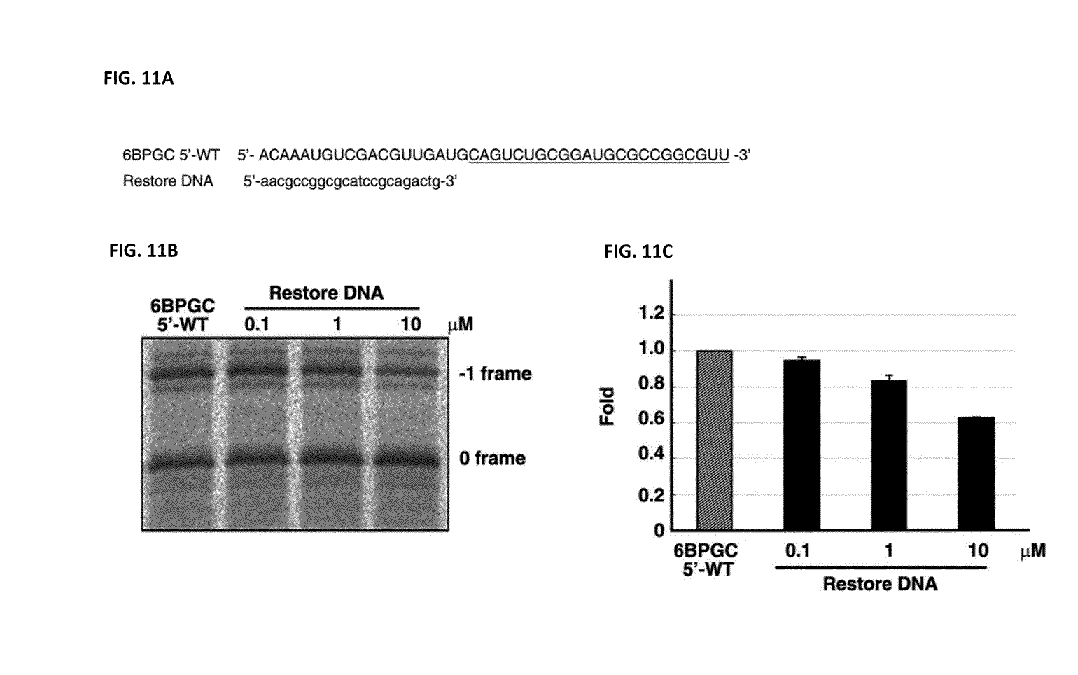

Addition of an antisense DNA (Restore DNA in FIG. 11A), with sequences complementary to the 3′-element of a defective attenuator 6BPGC5′-WT, led to the attenuation of −1 frameshifting efficiency of reporter containing 6BPGC5′-WT in a dose-dependent manner (FIGS. 11A, 11B and 11C), indicating that an upstream duplex is also capable of attenuating −1 PRF. These data also indicate that the reformed stem in the upstream hairpin during co-translational hairpin refolding is the functional determinant for reading-frame switch regulation.

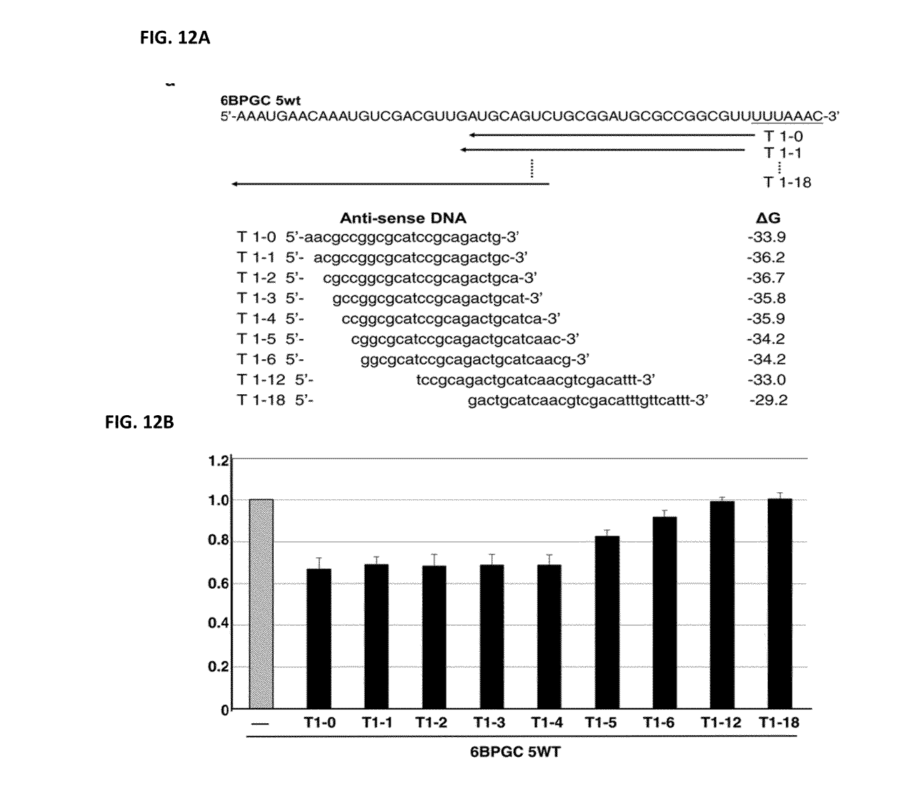

FIG. 12 shows the anti-sense DNAs complementary to sequences of a defective −1 PRF attenuator hairpin (6BPGC-5′ TW), and their analytic results of relative frameshifting activity in in vitro −1 PRF assay using reticulocyte lysate. As showed, attenuation efficiency is determined by duplex stability and its distance from the slippery site.

An Antisense-Mediated Upstream Duplex Provides an Alternative Approach to Inhibit −1 PRF Dependent Viral Pathogens.

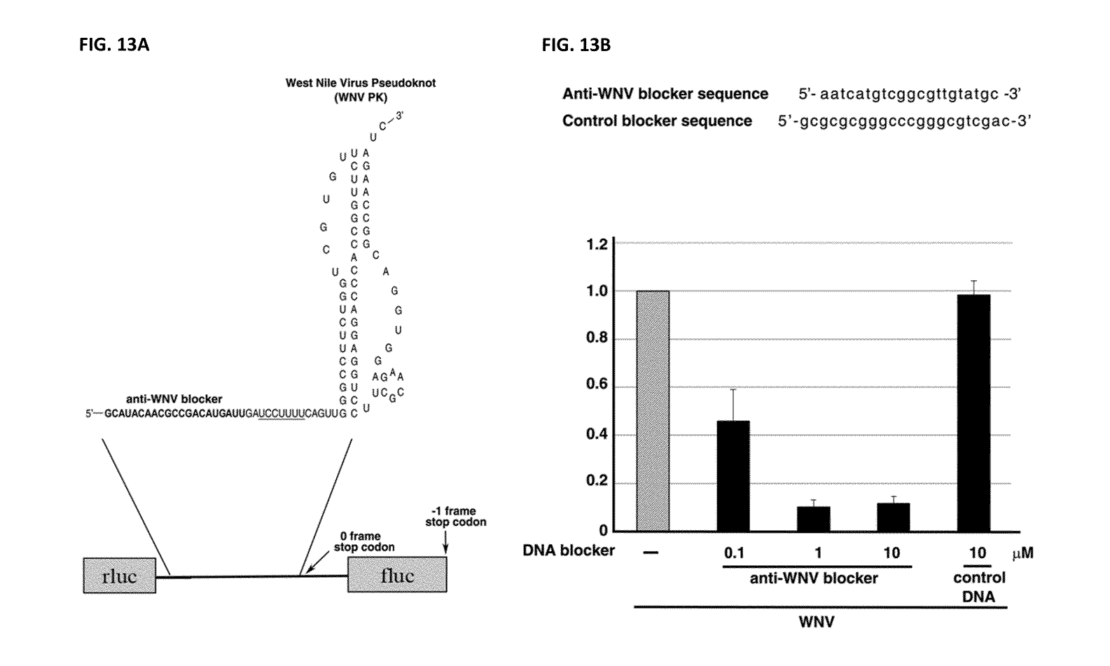

Based on this discovery, we designed an anti-sense DNA to target the sequences upstream of the −1 PRF frameshifting site in NS2A gene of West-Nile virus (WNV) and compared the −1 PRF inhibitory effects with an irrelevant oligonucleotide. We found that such an antisense approach efficiently reduced the −1 PRF activity of a reporter containing WN viral sequences, whereas an irrelevant oligonucleotide possessed no such activity (FIG. 13).

Theophylline-Dependent Control of Upstream +1 PRF Stimulator Hairpin Formation by an Upstream Theo-OFF2 Element to Down-Regulate +1 PRF in Yeast System.

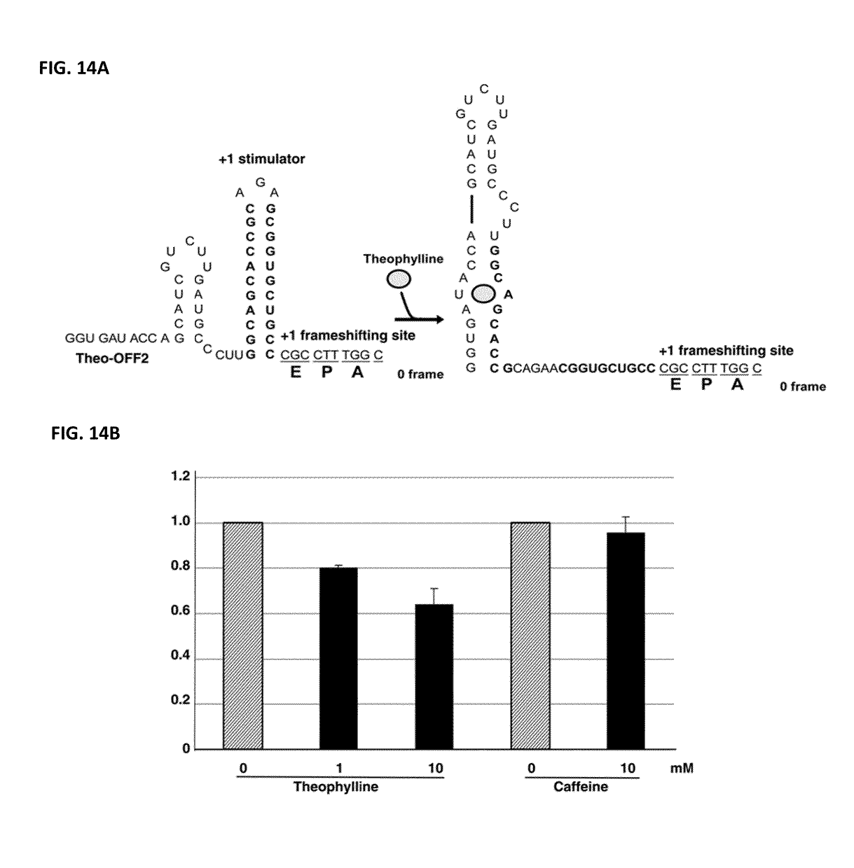

A CUUUGG sequence was used as the potential +1 frameshifting site to replace the spontaneous CUUAGG +1 frameshifting sequence in yeast. The theo-OFF2 element designed in FIG. 4 was then placed upstream of the CUUUGG sequence. As binding of theophylline to theophylline aptamer sequence should lead to the disruption of the hairpin upstream of frameshifting site (the upstream hairpin is a +1 frameshifting stimulator in contrast to being an attenuator for −1 PRF in FIG. 4), addition of theophylline led to the down-regulation of +1 PRF (FIG. 14).

As summarized in FIG. 1B, the present invention has disclosed the concept, design, and application of possible ligands in the regulation of upstream −1 PRF attenuator or +1 PRF stimulator that enable to explore the integration of metabolite-riboswitch interactions into translational regulation in eukaryotic cells, including plant and mammalian cells. Notably, the present invention demonstrates the ligand-dependent regulatory potential of a co-translational refolding RNA hairpin in ribosomal reading-frame switch control, side-stepping the need to look for a ligand-responsive pseudoknot.

Additionally, the present invention provides a unique way to regulate −1 PRF in a synergetic manner while being combined with a downstream ligand-responsive stimulator. The enhancement of regulatory dynamic range, by rational redesign of attenuator hairpin and replacing a potent stimulator with a weaker ligand-responsive stimulator (FIGS. 4, 6 and 7), also provides a platform for further improvement.

What is claimed is:

1. A method of regulating gene expression in a eukaryotic cell, the method comprising administrating a ligand-sensing RNA element to regulate the formation of regulatory hairpin upstream of programmed ribosomal frameshifting (PRF) site.

2. The method of claim 1, wherein the eukaryotic cell is a plant cell.

3. The method of claim 1, wherein the eukaryotic cell is a mammalian cell.

4. The method of claim 1, wherein the regulation of upstream PRF regulatory hairpin formation includes an enhancement of the formation of the hairpin structure.

5. The method of claim 1, wherein the regulation of upstream PRF regulatory hairpin formation includes an inhibition of the formation of the hairpin structure.

6. The method of claim 1, wherein the programmed ribosomal frameshifting is −1 PRF.

7. The method of claim 1, wherein the programmed ribosomal frameshifting is +1 PRF.

8. The method of claim 1, wherein the ligand is a molecule binding to the upstream PRF regulatory hairpin forming sequence in mRNA of the gene.

9. The method of claim 8, wherein the ligand is an antisense sequence complementary to the upstream PRF regulatory hairpin forming sequence in mRNA of the gene.

10. The method of claim 8, wherein the ligand is an RNA-binding protein binding to the upstream PRF regulatory hairpin forming sequence in mRNA of the gene.

11. The method of claim 8, wherein the ligand is an organic compound binding to the upstream PRF regulatory hairpin forming sequence in mRNA of the gene.

12. A method for regulating ribosome frameshifting efficiency in the protein translation of a eukaryotic cell, the method comprising contacting the eukaryotic cell with a molecule to inhibit or enhance the formation of a regulatory duplex element upstream of ribosomal frameshifting site.

13. The method of claim 12, wherein the eukaryotic cell is a plant cell.

14. The method of claim 12, wherein the eukaryotic cell is a mammalian cell.

15. The method of claim 12, wherein the molecule is an RNA-binding protein.

16. The method of claim 12, wherein the molecule is an organic compound.

17. The method of claim 12, wherein the molecule is an antisense sequence.
